(19) United States
(12) Patent Application Publication
(10) Pub. No.: US 2023/0010001 A1
(43) Pub. Date: Jan. 12, 2023

(54) PHOTOCROSSLINKED HYDROGELS BLENDED COMPOSITION, PREPARATION AND USE THEREOF

(71) Applicant: National Taiwan University, Taipei City (TW)

(72) Inventors: Po-Ling Kuo, Taipei City (TW); Shih-Hao Fu, Taipei City (TW)

(73) Assignee: National Taiwan University, Taipei City (TW)

(21) Appl. No.: 17/806,741

(22) Filed: Jun. 14, 2022

(30) Foreign Application Priority Data
Jun. 15, 2021 (TW) 110121760

(51) Int. Cl. C08J 3/075; C08J 3/12; C08J 3/28

(52) U.S. Cl. CPC C08J 3/075; C08J 3/12; C08J 3/28; C08J 2489/04

(57) ABSTRACT

The present invention discloses a partially crosslinked hydrogels blended composition with enhanced viscosity and yield stress, which is formed by the polymerization of one or more colloid monomers through crosslinking. The polymerization is initiated by a photoinitiator under irradiation of the light of a specific wavelength, which promotes crosslinking of the one or more colloid monomers. The hydrogels blended composition can be further crosslinked with one or more other colloid monomers through repeated excitation of the photoinitiator. The hydrogels blended composition can be polymerized into a gel upon re-irradiation, and can also be used as a biomaterial for wound repair, three-dimensional cell culture, personal nursing care, health care, medical and pharmaceutical applications.

### CROSS-REFERENCE TO RELATED APPLICATION

This application claims the benefit of Taiwan Patent Application No. 110121760, filed Jun. 15, 2021, which is incorporated by reference herein in its entirety.

### FIELD OF THE INVENTION

The present invention relates to a photopolymerized hydrogels blended composition. The hydrogels blended composition of the present invention can be repeatedly mixed with other uncrosslinked materials to adjust the colloidal properties. The polymerized hydrogels blended composition of the present invention can be applied to biomaterials for wound repair, three-dimensional cell culture, personal nursing care, health care, medical and pharmaceutical applications.

### BACKGROUND OF THE INVENTION

Hydrogels are colloidal polymers that swell in water, with characteristics such as high water content and high porosity, and can be applied to simulate natural biological tissues as synthetic biological materials, such as wound dressings, contact lenses, tissue engineering, hygiene products, drug delivery systems, biological drug carriers.

One of the gelation mechanisms of hydrogels is polymerization by crosslinking the functional groups of hydrophilic monomers with each other.

Another gelation mechanism of hydrogels is polymeric crosslinking of colloidal functional groups through the continuous reaction of free radicals, such as photopolymerization.

The crosslinking of hydrogel colloids can be regulated by changing environmental factors, such as changing the temperature, ionic strength, acid-base (pH) value of the colloids, thereby changing the bonding state of the colloids. These environmental factors also further affect the porosity and mechanical properties of the colloids

Clinically, hydrogel colloids can be used in wound dressing for drug delivery or cell therapy.

The clinical application of hydrogels for wound dressing can be covering the wounds with gels fabricated in advance, or by applying a hydrogel prepolymer solution to the wounds, and then polymerizing the gel. The latter is more potential for clinical application, because it provides better matching of the wound shape, and allows on-site customization easily of the dressing content.

However, commercially available photopolymerized hydrogel prepolymers suitable for cell therapy all have the problems of low viscosity and low yield stress at physiological temperature, and are prone to flowing away when applied to intestinal walls, blood vessels, or wounds on the non-horizontal side or uneven surface.

### SUMMARY OF THE INVENTION

The present invention discloses a hydrogels blended composition comprising: a hydrogel prepolymer comprising a photopolymerizable monomer and a photopolymerizable gel-forming component, wherein the photopolymerizable monomer and the photopolymerizable gel-forming component have not undergone photopolymerization reaction; a crosslinked hydrogel polymer, wherein the crosslinked hydrogel polymer refers to the hydrogel polymer produced by the photopolymerization reaction of the hydrogel prepolymer and a photoinitiator; and the photoinitiator.

The present invention discloses a method of preparing a hydrogels blended composition, comprising: providing a colloidal mixture, wherein the colloidal mixture comprises a hydrogel prepolymer comprising a photopolymerizable monomer and a photopolymerizable gel-forming component, wherein the photopolymerizable monomer and the photopolymerizable gel-forming component have not undergone photopolymerization reaction, and a crosslinked hydrogel polymer, wherein the crosslinked hydrogel polymer refers to the hydrogel polymer produced by the photopolymerization reaction of the hydrogel prepolymer and a photoinitiator; and irradiating the colloidal mixture with the light of a wavelength capable of photopolymerization, so that the colloidal mixture undergoes photocrosslinking polymerization to form a hydrogels blended composition.

The photoinitiator of the present invention refers to the photoinitiator with crosslinking activity that remains in a partially crosslinked hydrogel polymer. Additional photoinitiators can also be added.

Present invention discloses a hydrogels blended composition with high viscosity, high yield stress, good water absorption, and high cytocompatibility, which can significantly increase the viscosity, cohesion, and adhesion of the blended fluids at physiological temperature. Therefore, it can be applied to, including but not limited to, the repair, fixation, support, cell culture, and the carrier for biomaterials delivery, etc, of wounds on inclined and curved surface.

The hydrogels blended composition of the present invention contains functional groups that are still photopolymerizable, such as methacrylic groups and methacrylate groups, and still active photoinitiato. After the freeze-dried powder of the composition is mixed with an aqueous solution containing or not containing a photoinitiator, the mixture is irradiated with the light of the wavelength absorbed by the photoinitiator to solidify the mixture into a gel.

The partially crosslinked hydrogels blend composition is formed by mixing the hydrogels blend composition with a photoinitiator and irradiating with the light of a specific wavelength to initiate crosslinking polymerization.

The specific wavelength of the light for irradiation is determined by the characteristics of the photoinitiator, and the irradiation time can also be adjusted.

The partially crosslinked hydrogels blended composition after photocrosslinking photopolymerization can be further freeze-dried into colloidal granular powder, which has commercial value, expanded usage, and extended shelf life.

The granular powder can be prepared by photomask or microfluidic molding during photopolymerization, or by electrospinning into nanoparticles before photopolymerization, followed by freeze drying; or grinding the whole block freeze-dried hydrogels blended composition into powder.

The freeze-dried colloidal powder is easy to store. Since the active photoinitiators still remains in the colloidal powder, and it already contains functional groups that can undergo photopolymerization, a hydrogel can be formed again by dissolving the colloidal powder in an aqueous solution and irradiation with the light of specific wavelength corresponding to a specific photoinitiator.

The present invention discloses a recrosslinkable hydrogels blended composition comprising: a crosslinked hydrogel polymer, wherein the crosslinked hydrogel polymer refers to the hydrogel polymer produced by photopolymerization of a hydrogel prepolymer and a photoinitiator; and a buffer, wherein the crosslinked hydrogel polymer comprises a photoinitiator.

The present invention discloses a method of preparing a recrosslinkable hydrogels blended composition comprising: providing a crosslinked hydrogel polymer and a buffer, wherein the crosslinked hydrogel polymer refers to the hydrogel polymer produced by photopolymerization of a hydrogel prepolymer and a photoinitiator; and a buffer; dissolving the crosslinked hydrogel polymer in the buffer, followed by irradiating with light at a wavelength capable of photopolymerization to induce the photopolymerization crosslinking reaction of the crosslinked hydrogel polymer dissolved in the buffer to form a re-crosslinked hydrogel blended composition.

The partially-crosslinked hydrogels blended compositions of the present invention can be applied to wound repair, including but not limited to wounds on body surface, deep wounds such as those on intestinal walls, blood vessels, and the non-horizontal or curved wounds.

The wound dressing solution of the present invention obtained by mixing with the freeze-dried hydrogel powder can solve the above-mentioned problem of easy loss of the solution when coated on the wound due to its increased viscosity and yield stress.

In addition to being photocrosslinking with a hydrogel prepolymer by mixing with the prepolymer and another irradiation of the mixture, the freeze-dried hydrogel powder of the present invention can also be directly added to an aqueous solution of specific biological products. After mixing thoroughly, the mixture can be directly injected into the treatment target, which can be used as a therapeutic agent for repairing wounds or diseased tissue, cultivating three-dimensional cells, and improving the viscosity of biological products.

Aqueous solutions of biological products comprise pharmaceuticals, physiological saline, dextrose water injection, cell suspensions, exosomes, platelet-rich plasma, or mixtures of the above and a photopolymerizable hydrogel prepolymers solution, etc.

In detail, the aqueous solution of biological product and the freeze-dried hydrogel powder of appropriate percentage by weight are placed into an empty syringe. After thoroughly mixing, the mixture is pushed to the treatment target such as wound or diseased tissue by a plunger.

The term “appropriate percentage by weight” refers to a weight percentage that can increase the viscosity and yield stress of the mixture while maintaining considerable fluidity, in order to facilitate the smearing and retention of the mixture at the treatment target.

Through the above mixing steps, the viscosity, fluidity, and yield stress of the mixture can be adjusted to facilitate smearing, retention, or injection at the target, and the above-mentioned mechanical properties can be adjusted according to the condition of the treatment target.

Because the dried granular powder of the present invention has colloidal properties and also contains a photoinitiator, it can be re-gelled by photocuring.

In addition, the freeze-dried powder is easy to store, and already contains functional groups that can undergo photopolymerization, as well as photoinitiators that can still be activated, which can be used for making gels.

Therefore, the mixture after smearing can be photocured by irradiating with light of a wavelength absorbed by the photoinitiator in the powder.

The above photopolymerizable hydrogel prepolymer can be the same hydrogel component as the freeze-dried hydrogel powder, or a combination with other hydrogel prepolymers.

Through the above re-polymerization, the mechanical properties of the hydrogel such as the hardness can be adjusted, thus facilitating the colloid to engage with the target surface and conform to its geometry.

The freeze-dried hydrogel powder can also be mixed with a cell suspension and placed into a cell culture equipment, followed by being irradiated with the light of a wavelength absorbed by the photoinitiator in the powder to solidify for three-dimensional cell culture.

Due to the biocompatibility of the hydrogel colloidal polymers, the colloidal components that can undergo colloidal polymerization include but not limited to the following components: gelatin methacryloyl (GelMA), methacrylated hyaluronic acid, dextran-methacrylate, carboxymethylcellulose-methacrylate, 2-hydroxyethyl methacrylate, chitosan-gelatin methacrylate, methacrylated hydroxylbutyl chitosan, poly(ethylene glycol)methacrylate, poly(ethylene glycol)dimethacrylate, poly(ethylene glycol)diacrylate, carboxybetaine methacrylate, pullulan methacrylate, hyaluronic acid glycidyl methacrylate, 2-hydroxyethly methacrylate, 2-hydroxypropyl methacrylate, glycidyl methacrylate, sulfobetaine methacrylate.

In a preferred embodiment, the partially crosslinked colloidal polymer in the hydrogel of the present invention is composed of a photopolymerized gelatin methacryloyl (GelMA) polymer composed of gelatin and methacrylic anhydride.

In an embodiment of the present invention, the photopolymerizable monomer may be: gelatin, dextrin, carboxymethyl cellulose, hydroxyethyl methacrylate, hyaluronic acid, amylopectin, ethylene glycol, carboxybetaine, 2-hydroxyethyl ester, 2-hydroxypropyl ester, glycidyl ester, sulfobetaine, or a combination thereof.

In an embodiment of the present invention, the photopolymerizable gel-forming component may be: methacrylic anhydride, methyl acrylate, methacrylic acid, methacrylic ester, hyaluronic acid sodium salt, dextrin, carboxymethyl cellulose, hydroxyethyl methacrylate, 3-[[2-(methacryloyloxy)ethyl]dimethylamine]propionate, diacrylate, or a combination thereof.

In an embodiment of the present invention, the photoinitiator may be: lithium phenyl-2,4,6-trimethylbenzoylphosphinate and its salts, 2-Hydroxy-4′-(2-hydroxyethoxy)-2-methylpropiophenone, eosin-Y, diphenyl(2,4,6-trimethylbenzoyl)phosphine oxide, or a combination thereof.

In a preferred embodiment, the photoinitiator used in the hydrogel of the present invention is lithium phenyl-2,4,6-trimethylbenzoylphosphinate.

In a preferred embodiment, the irradiation time to activate the photoinitiator of the hydrogel of the present invention to form a gel is 60 seconds.

In an embodiment of the present invention, the buffer may be HEPES buffer, MES buffer, Bis-Tris buffer, citrate, ADA buffer, ACES buffer, PIPES buffer, imidazole/imidazole buffer, Bis-Tris propane buffer, maleic acid buffer, phosphate buffer, MOPSO buffer, BES buffer, MOPS buffer, TES buffer, DIPSO buffer, MOBS buffer, TAPSO buffer, HEPPSO buffer, POPSO buffer, EPPS (HEPPS) buffer, Tricine buffer, Gly-Gly buffer, Bicine buffer, HEPBS buffer, TAPS buffer, AMPD buffer, TABS buffer, AMPSO buffer, PIPPS buffer, methyl malonate, diethyl malonate, glycinamide hydrochloride buffer, or a combination thereof, or a buffer formulated according to any of the above buffers.

In a preferred embodiment, the buffer solution in which the hydrogel of the present invention dissolves is Dulbecco's Phosphate Buffered Saline (DPBS).

The hydrogels blended composition of the present invention may further comprise one or more drugs, active ingredients, bioactive materials, absorbent materials, or a combination thereof.

### BRIEF DESCRIPTION OF THE DRAWINGS

FIG. 1: Shear rate diagram of the freeze-dried hydrogel powder made of gelatin methacryloyl (GelMA) of various concentrations and mixed with buffer or gelatin methacryloyl prepolymer solution in a 1:6 weight ratio, compared with that of gelatin methacryloyl (GelMA) prepolymer solutions of different GelMA concentrations and that of pure water.

FIG. 2: Cytotoxicity profile of freeze-dried gelatin methacryloyl (GelMA) hydrogel powder evaluated by a Cell Counting Kit-8 (CCK8) kit.

FIG. 3: Images of live/dead cell staining of human placenta chorionic decidual-derived mesenchymal stem cells cultured in the re-gelled mixture of the freeze-dried gelatin methacryloyl (GelMA) hydrogel powder and buffer, and the quantitative results.

FIG. 4: Plot of shear stress versus shear rate for 10% powder blends, 20% GelMA prepolymer solution, and 10% powder blends plus 20% GelMA prepolymer solution.

FIG. 5: The storage modulus and loss modulus of the mixture of the freeze-dried hydrogel powder with buffer, and that of the re-gelled mixture after secondary UV irradiation measured with a rheometer.

FIG. 6: Appearance difference of the freeze-dried hydrogel powder mixture before and after secondary irradiation.

### DETAILED DESCRIPTION OF THE INVENTION

### Preparation of Freeze-Dried GelMA Solids

Gelatin and methacrylic anhydride were mixed in a 0.1M carbonate-bicarbonate buffer at a ratio of 10 g to 1 mL. The pH value of the mixture was adjusted to 7.4. After fully dialysis and purification with double distilled water, it was freeze-dried to form a white solid.

### Preparation of 10% GelMA Hydrogel

The freeze-dried GelMA solids were dissolved in phosphate buffer at 10% by weight, and 0.25% photoinitiator (lithium phenyl-2,4,6-trimethylbenzoylphosphinate) was added. After being heated to 60° C. in oven followed by sterile filtration, the mixture was irradiated with the ultraviolet light at the absorption wavelength of lithium phenyl-2,4,6-trimethylbenzoylphosphinate for 60 seconds to form a gel.

### Preparation of Freeze-Dried Hydrogel Powder

The 10% GelMA hydrogel was placed in a −20° C. refrigerator until freeze, followed by being freeze-dried with a freeze dryer. The resulting freeze-dried product was ground into powder and sieved to obtain the desired freeze-dried hydrogel powder.

Measurement of Viscosity Coefficient

The 10% freeze-dried hydrogel powder was mixed with phosphate buffer at 16.66% by weight and the viscosity of the mixture was measured by a rheometer. The result showed that the viscosity coefficient of the mixture was about 1 to 10 (Pa·s) in the shear rate ranging from 10 to 100 s-1. In contrast, within the same shear rate range, the viscosity coefficient of water was about 10-3 (Pa·s), and the viscosity coefficient of 20 wt % gelatin methacryloyl (GelMA) prepolymer solution was about 10-1 (Pa·s).

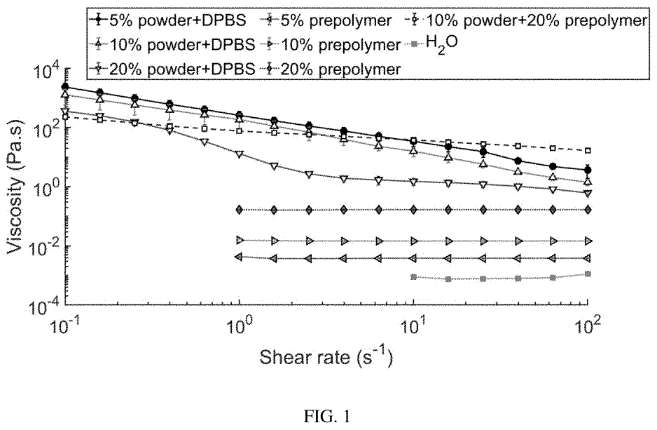

FIG. 1 showed the viscosity coefficient of 5% freeze-dried gel powder dissolved in DPBS, 10% freeze-dried gel powder dissolved in DPBS, 20% freeze-dried gel powder dissolved in DPBS, 5% gelatin methacryloyl (GelMA) hydrogel prepolymer solution, 10% gelatin methacryloyl (GelMA) hydrogel prepolymer solution, 20% gelatin methacryloyl (GelMA) hydrogel prepolymer solution, 10% freeze-dried gel powder plus 20% gelatin methacryloyl (GelMA) hydrogel prepolymer solution, and water measured by a rheometer (HR-2 system, TA Instruments) at 37° C. in a stress control manner.

The percent number in front of the group of freeze-dried gel powder dissolved in DPBS represented the concentration of the hydrogel before polymerization. All hydrogels were polymerized by irradiation for 60 seconds, and after lyophilization and grinding, the lyophilized hydrogel powder was mixed with DPBS (Dulbecco's phosphate-buffered saline) at a ratio of 1 g to 6000 μl. The group of 10% freeze-dried gel powder plus 20% gelatin methacryloyl (GelMA) hydrogel prepolymer solution was prepared by mixing freeze-dried hydrogel powder with 20% gelatin methacryloyl (GelMA) hydrogel prepolymer solution at a ratio of 1 g to 6000 μl. The number of samples in each group was 3.

As shown in FIG. 1, the groups of 5%, 10%, and 20% hydrogel prepolymer solution and water were all Newtonian fluids. In contrast, the mixtures added with freeze-dried gel powder were all non-Newtonian fluids and their viscosity decreases with increasing shear rate (shear thinning). The freeze-dried gel powder mixtures had higher viscosity than the hydrogel prepolymer solution at the same concentration as prepared. When the shear rate was between 10 and 100 per second, the viscosity of the freeze-dried hydrogel powder mixture was 500 to 40000 times that of water and 10 to 1500 times that of the 20% hydrogel prepolymer solution. After mixing the 20% hydrogel prepolymer solution with the 10% freeze-dried gel powder, the viscosity increased by 100 to 400 times.

Cytotoxicity Assay

Cytotoxicity of the freeze-dried hydrogel powder was examined using a Cell Counting Kit-8 (CCK8) kit. The test groups were as follows: a blank group using only cells and culture medium, three experimental groups in which the culture medium was mixed with the extract of the gelatin methacryloyl freeze-dried hydrogel powder dissolved in buffer with three weight volume ratios respectively, and a positive control group using bleach.

The method for obtaining the freeze-dried hydrogel powder extract was as follows: 10% gelatin methacryloyl freeze-dried hydrogel powder and culture medium were mixed in a weight-to-volume ratio of 1 gram to 6000 microliters, 2 grams to 6000 microliters, 0.5 grams to 6000 microliters and placed in a 37° C. incubator, followed by continuous extraction for 24 hours at a stirring speed of 100 rpm.

The cells used were NIH 3T3 fibroblasts cultured in a 96-well plate, and the cell amount was about 104 per well.

After incubation, the Cell Counting Kit-8 (CCK8) reagent was used to detect cell viability. The absorbance wavelength of the reagent was 450 nm. The number of samples in each group was 5.

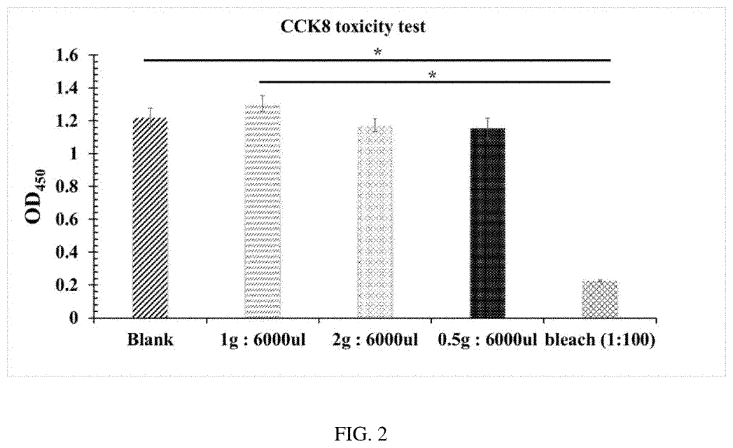

According to the results shown in FIG. 2, after culturing with different concentrations of the powder extract, the cell viability was close to that of the blank group, which means that the freeze-dried powder has no obvious toxicity to the cells.

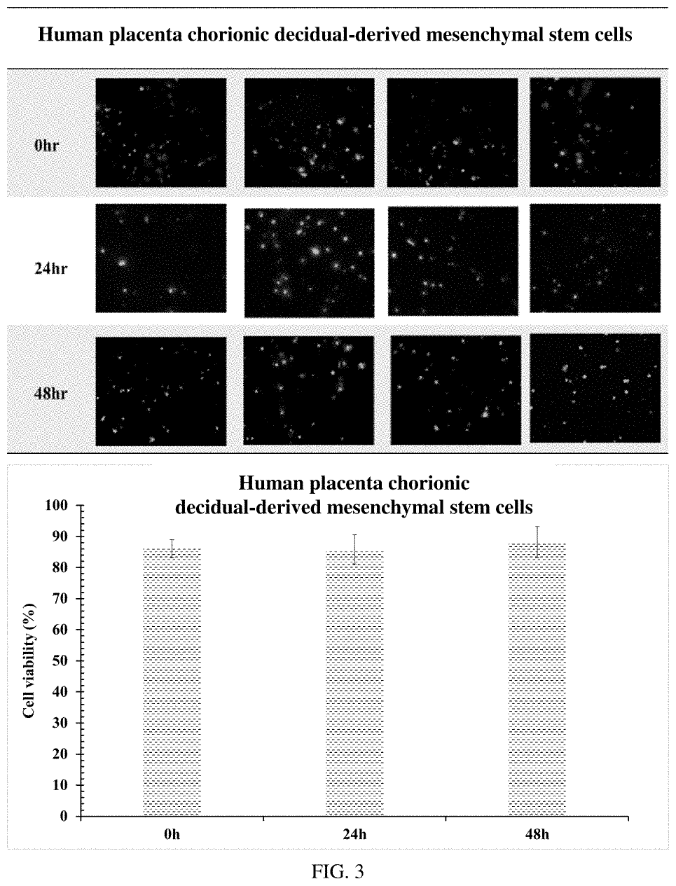

The experiment in FIG. 3 was as follows: 10% gelatin methacryloyl prepolymer solution was gelatinized by exposure to light for 60 seconds, followed by being prepared into freeze-dried hydrogel powder. The powder was mixed with DPBS solution in a weight-to-volume ratio of 1 g to 6000 μl. The resulting mixture was then mixed with 1×105 human placenta chorionic decidual-derived mesenchymal stem cells and spread evenly on a petri dish, which was irradiated with UV light for 30 seconds to from the gel again. After 0, 24, and 48 hours of incubation, cell viability was quantified using reagents of calcein AM and propidium iodide. The samples at each time point were from 3 independent experiments.

According to the results shown in FIG. 3, it was found that the cell viability was still more than 80% after 48 hours, indicating that the culture environment re-gelled with gelatin methacryloyl (GelMA) freeze-dried powder had no obvious cytotoxicity.

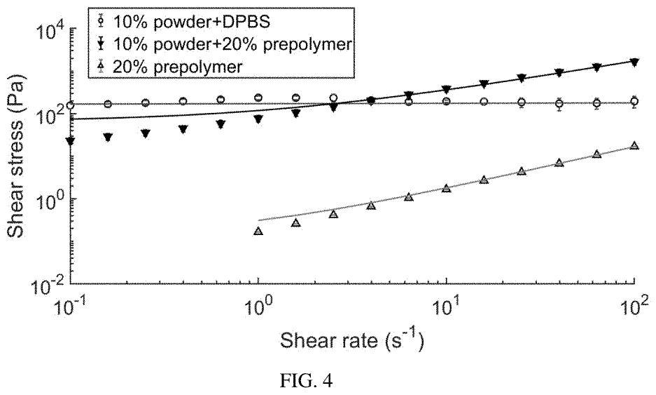

The experiment in FIG. 4 was as follows: The relations between shear stress and shear rate of 10% freeze-dried gel powder dissolved in DPBS, 20% gelatin methacryloyl prepolymer solution, and 10% powder plus 20% gelatin methacryloyl prepolymer solution were measured by a rheometer The percent number of the 10% powder represented the concentration of the hydrogel before polymerization. Hydrogels were polymerized by irradiation for 60 seconds, and after lyophilization and grinding, the lyophilized hydrogel powder was mixed with DPBS at a ratio of 1 g to 6000 μl. The percent number of the 10% powder plus 20% gelatin methacryloyl prepolymer solution represented the concentration of the hydrogel before polymerization. Hydrogels were polymerized by irradiation for 60 seconds, and after lyophilization and grinding, the lyophilized hydrogel powder was mixed with 20% gelatin methacryloyl prepolymer solution at a ratio of 1 g to 6000 μl. The number of samples in each test group was 3.

The data shown in FIG. 4 were then fitted with the Herschel-Bulkley model and the Bingham model respectively (curves in the figure) to estimate the yield stress of the 10% powder dissolved in DPBS, the 20% gelatin methacryloyl prepolymer solution, and the 10% powder plus 20% gelatin methacryloyl prepolymer solution were compared with each other. The higher the yield stress's number, the closer it is regarded to a solid, and the greater the cohesive force or cohesiveness.

According to the models, the yield stress of the 10% powder dissolved in DPBS, the 10% powder plus 20% gelatin methacryloyl prepolymer solution, and the 20% gelatin methacryloyl prepolymer solution were 41.5 Pa, 64.8 Pa, 0.14 Pa, respectively, indicating that the cohesive force of the freeze-dried hydrogel powder mixture was much greater than that of the prepolymer solution, which made it easier to stay on the inclined surface without running off.

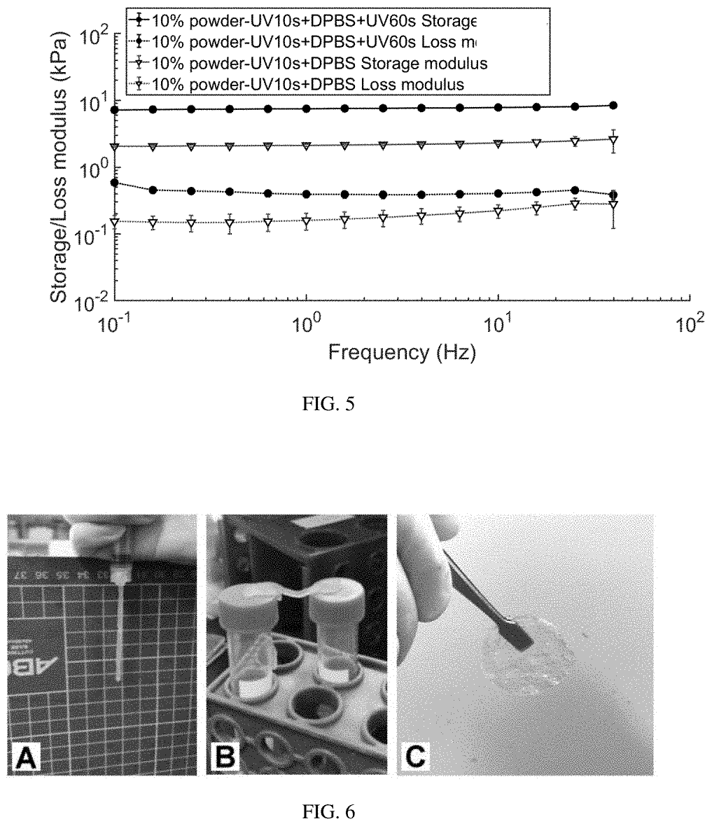

As shown in FIG. 5, the storage modulus and loss modulus of the freeze-dried hydrogel powder mixture before and after secondary UV irradiation was measured by a rheometer. The group of the 10% powder-UV10s+DPBS represented a 10% gelatin methacryloyl (GelMA) prepolymer solution that was irradiated with UV for 10 seconds to form a gel, followed by lyophilization, and then the lyophilized hydrogel powder was mixed with DPBS at a weight to volume ratio of 1 g to 6000 μl. to form a mixture. The group of the 10% powder-UV10s+DPBS+UV60s represented the above mixture that was squeezed onto a petri dish with a syringe and spread evenly before being irradiated with UV for an additional 60 seconds.

According to the results shown in FIG. 5, the storage modulus of the mixture after secondary irradiation was significantly higher than that of the mixture before the secondary irradiation, indicating that there might still be remaining photoinitiators and functional groups in the lyophilized hydrogel powder mixture, which could be further crosslinked by re-irradiation to strengthen the firmness of the mixture.

FIG. 6 illustrates the appearance difference of the freeze-dried hydrogel powder mixture before and after secondary irradiation. The freeze-dried hydrogel powder was prepared by irradiating a 10% gelatin methacryloyl (GelMA) prepolymer solution for 10 seconds and was dissolved in DPBS in a weight-to-volume ratio of 1 g to 6000 μL to yield the mixture. As the mixture was extruded through a syringe, as shown in FIG. 6 (A), the mixture remaining in the syringe was able to hold several centimeters of extrudate without breaking.

In FIG. 6 (B), the freeze-dried hydrogel powder mixture in FIG. 6 (A) was quickly extruded into a thread through a syringe and placed on the top of two centrifuge tubes. It was found that the cohesive force of the mixture was large enough so that the thread would not break in the middle.

In FIG. 6 (C), the mixture of FIG. 6 (A) was irradiated again for 60 seconds to obtain a gel which could be easily picked up with tweezers.

Although the present invention has been described and illustrated in sufficient detail to enable those skilled in the art to make and use it, various alternatives, modifications and improvements should be apparent without departing from the spirit and scope of the present invention.

1. A hydrogels blended composition comprising:
a hydrogel prepolymer comprising a photopolymerizable monomer and a photopolymerizable gel-forming component, wherein the photopolymerizable monomer and the photopolymerizable gel-forming component have not undergone photopolymerization reaction;
a crosslinked hydrogel polymer, wherein the crosslinked hydrogel polymer refers to the hydrogel polymer produced by the photopolymerization reaction of the hydrogel prepolymer and a photoinitiator; and
a photoinitiator

2. The hydrogels blended composition of claim 1, wherein the photoinitiator is the remaining photoinitiator in the crosslinked hydrogel polymer.

3. The hydrogels blended composition of claim 1, wherein the hydrogel prepolymer is selected from gelatin methacryloyl, methacrylated hyaluronic acid, dextran-methacrylate, carboxymethylcellulose-methacrylate, 2-hydroxyethyl methacrylate, chitosan-gelatin methacrylate, methacrylated hydroxylbutyl chitosan, poly(ethylene glycol)methacrylate, poly(ethylene glycol)dimethacrylate, poly(ethylene glycol)diacrylate, carboxybetaine methacrylate, pullulan methacrylate, hyaluronic acid glycidyl methacrylate, 2-hydroxyethly methacrylate, 2-hydroxypropyl methacrylate, glycidyl methacrylate, sulfobetaine methacrylate, or a combination thereof.

4. The hydrogels blended composition of claim 1, wherein the photopolymerizable monomer is selected from gelatin, dextrin, carboxymethyl cellulose, hydroxyethyl methacrylate, hyaluronic acid, amylopectin, ethylene glycol, carboxybetaine, 2-hydroxyethyl ester, 2-hydroxypropyl ester, glycidyl ester, sulfobetaine, or a combination thereof.

5. The hydrogels blended composition of claim 1, wherein the photopolymerizable gel-forming component is selected from methacrylic anhydride, methyl acrylate, methacrylic acid, methacrylic ester, hyaluronic acid sodium salt, dextrin, carboxymethyl cellulose, hydroxyethyl methacrylate, 3-[[2-(methacryloyloxy)ethyl]dimethylamine]propionate, diacrylate, or a combination thereof

6. The hydrogels blended composition of claim 1, wherein the hydrogels blended composition is used for preparing wound repair preparations, cultivating three-dimensional cells, or improving the viscosity or yield stress of biological products.

7. A method of preparing a hydrogels blended composition, comprising:
providing a colloidal mixture, wherein the colloidal mixture comprises:
a hydrogel prepolymer comprising a photopolymerizable monomer and a photopolymerizable gel-forming component, wherein the photopolymerizable monomer and the photopolymerizable gel-forming component have not undergone photopolymerization reaction, and a crosslinked hydrogel polymer, wherein the crosslinked hydrogel polymer refers to the hydrogel polymer produced by the photopolymerization reaction of the hydrogel prepolymer and a photoinitiator; and
irradiating the colloidal mixture with light at a wavelength capable of photopolymerization, so that the colloidal mixture undergoes photopolymerization crosslinking reaction to form a hydrogels blended composition.

8. The method of claim 7, wherein the crosslinked hydrogel polymer comprises a photoinitiator.

9. The method of claim 7, wherein the colloidal mixture is further freeze-dried to become powder.

10. A recrosslinkable hydrogels blended composition comprising:
a crosslinked hydrogel polymer, wherein the crosslinked hydrogel polymer refers to the hydrogel polymer produced by photopolymerization of a hydrogel prepolymer and a photoinitiator; and a buffer.

11. The recrosslinkable hydrogels blended composition of claim 10, wherein the crosslinked hydrogel polymer comprises a photoinitiator.

12. The recrosslinkable hydrogels blended composition of claim 10, wherein the hydrogel prepolymer is selected from gelatin methacryloyl, methacrylated hyaluronic acid, dextran-methacrylate, carboxymethylcellulose-methacrylate, 2-hydroxyethyl methacrylate, chitosan-gelatin methacrylate, methacrylated hydroxylbutyl chitosan, poly(ethylene glycol)methacrylate, poly(ethylene glycol)dimethacrylate, poly(ethylene glycol)diacrylate, carboxybetaine methacrylate, pullulan methacrylate, hyaluronic acid glycidyl methacrylate, 2-hydroxyethly methacrylate, 2-hydroxypropyl methacrylate, glycidyl methacrylate, sulfobetaine methacrylate, or a combination thereof.

13. The recrosslinkable hydrogels blended composition of claim 10, wherein the buffer is selected from HEPES buffer, MES buffer, Bis-Tris buffer, citrate, ADA buffer, ACES buffer, PIPES buffer, imidazole/imidazole buffer, Bis-Tris propane buffer, maleic acid buffer, phosphate buffer, MOPSO buffer, BES buffer, MOPS buffer, TES buffer, DIPSO buffer, MOBS buffer, TAPSO buffer, HEPPSO buffer, POPSO buffer, EPPS (HEPPS) buffer, Tricine buffer, Gly-Gly buffer, Bicine buffer, HEPBS buffer, TAPS buffer, AMPD buffer, TABS buffer, AMPSO buffer, PIPPS buffer, methyl malonate, diethyl malonate, glycinamide hydrochloride buffer, or a combination thereof.

14. The recrosslinkable hydrogels blended composition of claim 10, wherein the recrosslinkable hydrogels blended composition is used for preparing wound repair preparations, cultivating three-dimensional cells, or improving the viscosity or yield stress of biological products.

15. A method of preparing a recrosslinkable hydrogels blended composition comprising:
providing a crosslinked hydrogel polymer and a buffer, wherein the crosslinked hydrogel polymer refers to the hydrogel polymer produced by photopolymerization of a hydrogel prepolymer and a photoinitiator; and a buffer,
dissolving the crosslinked hydrogel polymer in the buffer; and irradiating with light at a wavelength capable of photopolymerization to induce the photopolymerization crosslinking reaction of the crosslinked hydrogel polymer dissolved in the buffer to form a re-crosslinked hydrogels blended composition.

16. The method of claim 15, wherein the recrosslinkable hydrogels blended composition is further freeze-dried to become powder.
